(19) United States
(12) Patent Application Publication
(10) Pub. No.: US 2009/0048339 A1
(43) Pub. Date: Feb. 19, 2009

(54) COMPOSITIONS OF CIS-9, TRANS-11 CONJUGATED LINOLEIC ACID AND VACCENIC ACID AND USES THEREOF

(75) Inventors: Rupinder Kaur Kanwar, Geelong (AU); Geoffrey Wayne Krissansen, Auckland (NZ); Peter Nigel Black, Auckland (NZ); Alastair Kenneth Hugh MacGibbon, Palmerston North (NZ)

Correspondence Address: KNOBBE MARTENS OLSON & BEAR LLP, 2040 MAIN STREET, FOURTEENTH FLOOR, IRVINE, CA, 92614, US

(73) Assignees: FONTERRA CORPORATE RESEARCH AND DEVELOPMENT LIMITE, Auckland (NZ); FONTERRA LIMITED, Auckland (NZ); AUCKLAND UNISERVICES LIMITED, Auckland (NZ)

(21) Appl. No.: 12/093,307

(22) PCT Filed: Nov. 10, 2006
(86) PCT No.: PCT/NZ2006/000289

§ 371 Date: Oct. 13, 2008

(30) Foreign Application Priority Data
Nov. 10, 2005 (NZ) 543486

(51) Int. Cl. A61K 31/201; A61P 11/08; A61P 11/06; A61P 11/12; A61P 17/00; A61P 17/06

(52) U.S. Cl. 514/560

(57) ABSTRACT

The invention relates to use of the cis-9, trans-11 isomer of conjugated linoleic acid or a salt or ester thereof (cis-9, trans-11 CLA) and vaccenic acid or a salt or ester thereof (VA) to treat or prevent conditions associated with one or more of leukocyte infiltration, eosinophilia, airway remodelling, bronchoconstriction, mucus hypersecretion, and lung and skin inflammation. The present invention also relates to a composition comprising cis-9, trans-11 CLA and VA and use of the composition to treat or pre-vent conditions associated with one or more of leukocyte infiltration, eosinophilia, airway remodelling, bronchoconstriction, mucus hypersecretion, and lung and skin inflammation. In particular, the medicinal uses, compositions and methods of the invention may be used to treat or prevent conditions such as asthma and dermatitis, and related disorders.

### FIELD OF THE INVENTION

The present invention relates to use of the cis-9, trans-11 isomer of conjugated linoleic acid or a salt or ester thereof (cis-9, trans-11 CLA) and vaccenic acid (trans 11-octadecenoic acid) or a salt or ester thereof (VA) to treat or prevent conditions associated with one or more of leukocyte infiltration, eosinophilia, airway remodelling, bronchoconstriction, mucus hypersecretion, and lung and skin inflammation. The present invention also relates to a composition comprising cis-9, trans-11 CLA or a salt or ester thereof and VA or a salt or ester thereof and use of the composition to treat or prevent conditions associated with one or more of leukocyte infiltration, eosinophilia, airway remodelling, bronchoconstriction, mucus hypersecretion and lung and skin inflammation. In particular, the medicinal uses, compositions and methods of the invention may be used to treat or prevent conditions such as asthma and dermatitis, and related disorders.

### BACKGROUND

Persons with atopy have a genetic predisposition to produce IgE antibodies against common environmental allergens, and often suffer from one or more atopic diseases including allergic rhinitis, asthma, and atopic eczema (Kay, 2001). Atopic individuals have an exaggerated response to allergen characterized by elevated levels of IgE antibodies, and their T cells respond to allergen by producing type 2 helper (Th2) cytokines, including interleukin-4 (IL-4), IL-5, IL-9 and IL-13 rather than the type I helper (Th1) cytokines IL-2 and interferon-gamma (IFN-garnma) that typify the normal response.

Exposure of a person with atopy to allergen can lead to an immediate hypersensitivity reaction in which a complex of allergen, IgE, and FcεRI on the surface of mast cells triggers the release of histamine, tryptase, and the lipid mediators leukotrienes, prostaglandins, and platelet-activating factor. The leukotrienes C4, D4, and E4 cause the contraction of smooth muscles, vasodilatation, increased vascular permeability, and hypersecretion of mucus. Tryptase activates a signalling pathway that leads to the upregulation of cell adhesion molecules on endothelial and epithelial cells that selectively attract eosinophils and basophils. In the subsequent late-phase reaction, eosinophils and neutrophils accumulate in the lung, followed by CD4+ T cells. Late-phase reactions can be induced in the absence of immediate hypersensitivity indicating T cells alone are sufficient to initiate narrowing of the airways in patients with allergic asthma.

Increased numbers of eosinophils is a hallmark of allergic disease, and eosinophils are enriched up to 100-fold in the airways of asthmatic subjects. A recent review reported that there is a broad correlation between the degree of eosinophilia and disease severity. Eosinophils are a characteristic feature of seasonal and perennial rhinitis (Christodoulopoulus, et al., 2000) and nasal polyposis (Lamblin, et al., 1999). There are increased numbers of eosinophils in atopic dermatitis, and deposition of eosinophil basic proteins in the affected skin (Erjefalt, et al., 1999). Degranulating eosinophils can injure mucosal surfaces by releasing toxic basic proteins, cysteinyl leukotrienes, and platelet activating factor which are thought to cause bronchospasm; and impair M2 muscarinic receptors responsible for controlling cholinergic responses. They have been proposed to play pathogenic roles in asthma, nasal polyposis, allergic rhinitis, and eosinophilic pneumonia (Lieferman, 1989; Gleich, et al., 1989).

Asthma attacks are triggered by the binding of inhaled allergens to IgE antibodies on the surfaces of sensitized mast cells in the lungs. Binding triggers mast cell degranulation and release of histamine and leukotrienes. These molecules cause the smooth muscle cells of the bronchi to contract, narrowing the lumen of the bronchi, attract inflammatory cells, especially eosinophils, and mediate mucus production. Existing medicines that are mast cell stabilisers inhibit immediate allergic responses but are not effective in treating chronic asthma. A medicine that inhibits mediator release from mast cells is unlikely to be an effective treatment for asthma unless it can be shown to have some other activity e.g. as a bronchodilator or inhibitor of eosinophilic inflammation.

Inhaled corticosteroids are now the recommended first-line therapy for asthma, as they improve lung function, decrease symptoms, reduce exacerbations, and can prevent more than half of all hospitalizations due to asthma (Suissa, et al., 2001). They are effective at reducing morbidity and mortality due to asthma, but they have to be regularly inhaled to remain effective. Inhaled corticosteroids are in some cases being prescribed for asthma at inappropriately high doses, with the potential to cause adverse effects such as osteoporosis, cataracts and adrenal suppression (Macdessi et al., 2003). A variety of therapeutic agents have been administered to asthma patients because of their steroid-sparing effect, including anti-IgE antibodies (Milgrom et al., 2001), leukotriene receptor antagonists (Frew et al., 2001), gold and methotrexate (Niven et al., 2003). Steroid-resistant asthma in which the patient derives reduced benefit from steroid use, is a serious medical challenge, and requires the delivery of non-steroidal anti-asthmatic drugs (Thomas et al., 1999).

The Western lifestyle is believed to be a contributing factor to the risk of developing asthma. Diets have changed significantly since we led a more pastoral existence. Epidemiological studies have suggested a beneficial effect of consuming oily fish (Hodge et al., 1996), however the results of intervention studies with fish oil has been inconsistent. A reduction in the levels of inflammatory mediators associated with asthma has been reported with dietary interventions such as administration of oils containing a combination of gamma-linolenic acid and eicosapentaenoic acid (EPA), normally derived from fish (Spector et al., 2003). Dietary supplementation with fish oil rich in EPA and docosahexaenoic acid (DHA) has been reported to be beneficial for children with bronchial asthma (Nagakura et al., 2000). A lipid extract from the New Zealand green-lipped mussel (Perna canaliculus) rich in the omega 3 fatty acids DHA and EPA reportedly decreased daytime wheeze, the concentration of exhaled H₂O₂, and increased morning peak expiratory flow in asthma patients (Emelyanov et al., 2002). A number of other studies have not shown any benefit from treatment with fish oil (Woods et al., 2002).

A recent study investigated the relationship between food consumption and asthma symptoms in 2978 pre-school children followed prospectively. It reported that the frequent consumption of products containing milk fat was associated with a reduced risk of asthma symptoms (Wijga et al., 2003). A number of other studies have suggested that consumption of dairy products can protect against the development of allergic sensitisation or atopic disease, and that conversely that polyunsaturated fat may be deleterious (Bolte et al., 2001; Dunder et al., 2001; von Mutius et al., 1998; Haby et al., 2001).

Milkfat contains a number of bioactive fatty acids. The most extensively studied fatty acid from milk is conjugated linoleic acid (CLA), which has been reported to exhibit a number of health benefits (Parodi, 2002). The tracheae of guinea pigs fed synthetic CLA enriched in t-10, c-12 isomer for two weeks reportedly displayed reduced contraction to allergen, which corresponded with increased release of prostaglandin E2 (PGE2) (International Patent Application WO 97/32008). In contradiction, the same authors reported in two subsequent papers that feeding of an approximately equal mixture of synthetic cis-9, trans-1 and trans-10, cis-12 isomers of CLA reduced allergen-induced histamine and release of PGE2 from allergen sensitized guinea pig tracheae (Whigham et al., 2001; Whigham et al., 2002), but did not affect allergen-induced tracheal contractions (Whigham et al., 2001). WO 2005/107736 reports that milk fat enriched with cis-9, trans-11 CLA is useful to treat or prevent conditions associated with one or more of leukocyte infiltration, eosinophilia, airway remodelling, bronchoconstriction and mucus hypersecretion, and is hereby incorporated by reference.

An exhaustive analysis of the published data on the influence of synthetic seed-derived CLA on immune function reported that supplementation of the diet with CLA is not recommended (Kelley et al., 2003). The synthetic cis-9, trans-11 CLA isomer appears relatively benign, whereas in contrast, the synthetic trans-10, cis-12 isomer has been shown to alter body fat mass, increase the fat content of several tissues, increase circulating insulin, and increase the saturated fatty acid content of adipose tissue and muscle (Kelley et al., 2003). In addition, it has been reported to cause a dramatic enlargement of the liver with steatosis when fed to mice at 0.4% w/w for 4 weeks (Clement et al., 2002). Trans-10, cis-12 CLA has also been shown to have deleterious effects in man (Risers et al., 2002). This latter study showed that trans-10, cis-12 CLA aggravated insulin resistance and increased CRP and 8-iso-prostane which is a marker of oxidative stress.

Vaccenic acid (trans 11-octadecenoic acid; VA), a trans fatty acid produced in the rumen of ruminants, is a major component of milk fat, constituting 1.7% (range: 0.4-4%) of the total fatty acid content (Precht, et al., 1996). VA is converted to cis-9, trans-11 CLA in the tissues of mice (Santora, et al., 2000) and humans (Turpeinen, et al., 2003) by delta(9)-desaturase.

To date, VA has not been examined in respect of its ability to attenuate inflammatory or allergic diseases. Its potential interaction with CLA has been reported in the cancer field. Banni et al. (2001) demonstrated that feeding rats VA increased the tissue concentrations of cis-9, trans-11 CLA, which has anti-carcinogenic properties. There was a corresponding reduction in the number of premalignant mammary lesions after exposure to a chemical carcinogen. Corl et al. (2003) extended these findings by demonstrating an additive effect for dietary CLA and VA. The combination caused a dose-dependent increase in the accumulation of CLA in the mammary fat pad and a parallel reduction in tumor number and incidence. Thus, the conversion of VA to cis-9, trans-11 CLA is as important for cancer prevention as is the dietary concentration of cis-9, trans-11 CLA. The effects of cis-9, trans-11 CLA on immune cell function have been widely investigated (reviewed by O'Shea et al., 2004). By contrast the immunomodulatory effects of VA are as yet unexplored. The cis-9, trans-11 CLA isomer is able to induce the apoptosis of Jurkat T cells, but VA is without effect (Bergamo et al., 2005), suggesting cis-9, trans-11 CLA and VA exert distinct effects on immune cells. Trans fatty acids have been shown to increase inflammation, and in particular the production of inflammatory cytokines (Wu, 2004; Lopez-Garcia et al., 2005; Mozaffarian et al., 2004).

In the treatment of cancer it was argued that VA may exert its effects independently of cis-9, trans-11 CLA, as evidenced by the fact that VA was able to modestly inhibit the growth of HT-29 human colon cancer cells compared with stearic acid (Awad, et al., 1995). However this finding could not be reproduced by a different research group (Lampen, et al., 2005).

Allergic dermatitis (also referred to as atopic dermatitis or eczema) is a skin inflammation caused by contact with a substance to which the affected person is allergic (Boguniewicz et al., 2006). The symptoms are a dry, itchy scaly rash, which commonly develops on the scalp, cheeks, and elbows, eyelids, neck, elbow creases, and back of knees. Allergic dermatitis is one of the most common skin diseases, particularly in infants and children. Topical corticosteroids are most commonly used to treat dermatitis, but they have their problems. They can increase the risk of skin thinning and other side effects, including headache, indigestion, increased appetite, restlessness, and increased risk of infection. The face is especially sensitive to thinning of the skin. Use of topical corticosteroids on the face can result in enlarged blood vessels (telangiectasias), bruising, acne, and stretch marks (striae). There is room for therapies that exhibit fewer side-effects, and which can be routinely applied to the face. In the only previous reported study of the effects of CLA on dermatitis, CLA (80% purity, c9, t11 and t10, c12 ratio 1:1) given at the dosage of 90 mg/kg per day did not improve the clinical signs of canine atopic dermatitis (Noli et al., 2006), but the cis-9, trans-11 CLA isomer was not tested alone.

It would therefore be desirable to provide an improved means for treating or preventing conditions associated with one or more of leukocyte infiltration, eosinophilia, airway remodelling, bronchoconstriction, mucus hypersecretion, and lung and skin inflammation, that overcomes or ameliorates problems associated with reported treatments or to at least provide the public with a useful choice.

### SUMMARY OF THE INVENTION

Accordingly, in one aspect the present invention relates to consisting essentially of, or consisting of cis-9, trans-11 conjugated linoleic acid (CLA) or a salt or ester thereof and vaccenic acid (VA) or a salt or ester thereof.

In another aspect the present, invention relates to a composition consisting essentially of, or consisting of about 1% to about 99% by weight cis-9, trans-11 CLA or a salt or ester thereof and about 99% to about 1% by weight VA or a salt or ester thereof.

In another aspect the present invention relates to a composition enriched with cis-9, trans-11 CLA or a salt or ester thereof and VA or a salt or ester thereof wherein the composition is a food, drink, food additive, drink additive, dietary supplement, nutritional product, medical food or nutraceutical. In another aspect the present invention relates to a first composition enriched with a second composition consisting essentially of, or consisting of cis-9, trans-11 CLA or a salt or ester thereof and VA or a salt or ester thereof wherein the composition is a food, drink, food additive, drink additive, dietary supplement, nutritional product, medical food or nutraceutical.

In another aspect the present invention relates to a composition comprising, consisting essentially of, or consisting of about 5% to about 30% by weight cis-9, trans-11 CLA or a salt or ester thereof and about 95% to about 70% by weight VA or a salt or ester thereof.

In another aspect the present invention relates to a composition comprising, consisting essentially of, or consisting of cis-9, trans-11 CLA or a salt or ester thereof and VA or a salt or ester thereof in a ratio of about 0.5:9.5 to about 3:7 by weight, the composition comprising at least about 7% by weight cis-9, trans-11 CLA or a salt or ester thereof based on the weight of the composition.

In another aspect the present invention relates to a composition comprising, consisting essentially of, or consisting of cis-9, trans-11 CLA or a salt or ester thereof and VA or a salt or ester thereof in a ratio of about 1:12 to about 1:6 by weight, the composition comprising at least about 7% by weight cis-9, trans-11 CLA or a salt or ester thereof based on the weight of the composition.

In another aspect the present invention relates to a pharmaceutical composition consisting essentially of, or consisting of cis-9, trans-11 CLA or a salt or ester thereof, VA or a salt or ester thereof and a pharmaceutically acceptable carrier.

In another aspect the present invention relates to a pharmaceutical composition consisting essentially of, or consisting of cis-9, trans-11 CLA or a salt or ester thereof, VA or a salt or ester thereof, a pharmaceutically acceptable carrier and optionally one or more agents selected from bronchodilators, anticholinergic agents and anti-inflammatory agents.

In another aspect the present invention relates to pharmaceutical composition comprising, consisting essentially of, or consisting of cis-9, trans-11 CLA or a salt or ester thereof, VA or a salt or ester thereof, and a pharmaceutically acceptable carrier, wherein the composition comprises about 1% to about 99% by weight cis-9, trans-11 CLA or a salt or ester thereof and about 99% to about 1% by weight VA or a salt or ester thereof based on the combined weight of the cis-9, trans-11 CLA or salt or ester thereof and the VA or salt or ester thereof.

In another aspect the present invention relates to pharmaceutical composition comprising, consisting essentially of, or consisting of cis-9, trans-11 CLA or a salt or ester thereof, VA or a salt or ester thereof, and a pharmaceutically acceptable carrier, wherein the composition comprises about 5% to about 30% by weight cis-9, trans-11 CLA or a salt or ester thereof and about 95% to about 70% by weight VA or a salt or ester thereof based on the combined weight of the cis-9, trans-11 CLA or salt or ester thereof and the VA or salt or ester thereof.

In another aspect the present invention relates to use of cis-9, trans-11 CLA or a salt or ester thereof and VA or a salt or ester thereof in the manufacture of a composition for treating or preventing a condition associated with one or more of leukocyte infiltration, eosinophilia, airway remodelling, bronchoconstriction, mucus hypersecretion, and lung and skin inflammation. In one embodiment the condition is selected from the conditions listed below including atopic conditions, eosinophilias and Th2-mediated conditions. In one embodiment the condition is asthma or dermatitis.

In another aspect the present invention relates to use of cis-9, trans-11 CLA or a salt or ester thereof and VA or a salt or ester thereof in the manufacture of a composition for treating or preventing a condition associated with one or more of leukocyte infiltration, eosinophilia, airway remodelling, bronchoconstriction, mucus hypersecretion and lung and skin inflammation, wherein the composition consists essentially of, or consists of cis-9, trans-11 CLA or a salt or ester thereof and VA or a salt or ester thereof and optionally one or more agents selected from bronchodilators, anticholinergic agents and anti-inflammatory agents.

In another aspect the present invention relates to use of cis-9, trans-11 CLA or a salt or ester thereof and VA or a salt or ester thereof in the manufacture of a medicament for treating or preventing a condition associated with one or more of leukocyte infiltration, eosinophilia, airway remodelling, bronchoconstriction, mucus hypersecretion and lung and skin inflammation, wherein the medicament consists essentially of, or consists of cis-9, trans-11 CLA or a salt or ester thereof, VA or a salt or ester thereof, a pharmaceutically acceptable carrier, and optionally one or more agents selected from bronchodilators, anticholinergic agents and anti-inflammatory agents.

In another aspect the present invention relates to use of cis-9, trans-11 CLA and VA in the manufacture of a composition for treating or preventing with steroid sparing effect a condition associated with one or more of leukocyte infiltration, eosinophilia, airway remodelling, bronchoconstriction, mucus hypersecretion and lung and skin inflammation. In one embodiment the condition is a steroid-dependent condition including corticosteroid dependent asthma, severe eczema and eosinophilic disorders including eosinophilic gastroenteritis, eosinophilic pneumonia and hyper-eosinophilic syndrome.

In another aspect the present invention relates to a method of treating or preventing a condition associated with one or more of leukocyte infiltration, eosinophilia, airway remodelling, bronchoconstriction, mucus hypersecretion, and lung and skin inflammation comprising administering cis-9, trans-11 CLA or a salt or ester thereof and VA or a salt or ester thereof separately, simultaneously or sequentially to a subject in need thereof. In one embodiment the condition is selected from the conditions listed below including atopic conditions, eosinophilias and Th2-mediated conditions. In one embodiment the condition is asthma or dermatitis. In one embodiment the method comprises administering a composition of the invention to a subject in need thereof.

In another aspect the present invention relates to a method for treating or preventing with steroid sparing effect a condition associated with one or more of leukocyte infiltration, eosinophilia, airway remodelling, bronchoconstriction, mucus hypersecretion, and lung and skin inflammation comprising administering cis-9, trans-11 CLA or a salt or ester thereof and VA or a salt or ester thereof separately, simultaneously or sequentially to a subject in need thereof. In one embodiment the condition is a steroid-dependent condition including corticosteroid dependent asthma, severe eczema and eosinophilic disorders including eosinophilic gastroenteritis, eosinophilic pneumonia and hyper-eosinophilic syndrome. In one embodiment the method comprises administering a composition of the invention to a subject in need thereof.

In another aspect the present invention relates to a product containing cis-9, trans-11 CLA or a salt or ester thereof and VA or a salt or ester as a combined preparation for simultaneous, separate or sequential use in therapy of a condition associated with one or more of leukocyte infiltration, eosinophilia, airway remodelling, bronchoconstriction, mucus hypersecretion, and lung and skin inflammation.

The following embodiments may relate to any of the above aspects.

In one embodiment the composition consists essentially of cis-9, trans-11 CLA or a salt or ester thereof and VA or a salt or ester thereof. In another embodiment the composition consists of cis-9, trans-11 CLA or a salt or ester thereof and VA or a salt or ester thereof. In a further embodiment the composition comprises, consists essentially of or consists of cis-9, trans-11 CLA or a salt or ester thereof, VA or a salt or ester thereof and an agent selected from bronchodilators, anticholinergic agents and anti-inflammatory agents. In yet another embodiment the composition comprises, consists essentially of or consists of cis-9, trans-11 CLA or a salt or ester thereof, VA or a salt or ester thereof and an agent selected from bronchodilators, anticholinergic agents and anti-inflammatory agents, and optionally a pharmaceutically acceptable carrier.

In one embodiment the composition further comprises one or more agents selected from bronchodilators, anticholinergic agents and anti-inflammatory agents. Useful bronchodilators include but are not limited to beta-2 agonists; anticholinergic agents include but are not limited to antimuscarinic agents and antinicotinic agents; and anti-inflammatory agents include but are not limited to inhaled steroids, intranasal steroids, steroid creams and ointments, oral steroids, leukotriene receptor antagonists, leukotriene antagonists and 5-lipoxygenase inhibitors.

In one embodiment the composition is substantially free of the trans-10, cis-12 CLA isomer. In another embodiment it is provided that the composition is not milk or milk fat.

In one embodiment the composition comprises about 0.1% to about 99.9% by weight cis-9, trans-11 CLA or a salt or ester thereof and about 99.9% to about 0.1% by weight VA or a salt or ester thereof. In another embodiment the composition comprises about 1% to about 99% by weight cis-9, trans-11 CLA or a salt or ester thereof and about 99% to about 1% by weight VA or a salt or ester thereof. In yet another embodiment the composition comprises about 5% to about 95% by weight cis-9, trans-11 CLA or a salt or ester thereof and about 95% to about 5% by weight VA or a salt or ester thereof.

In one embodiment the composition comprises at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight cis-9, trans-11 CLA or a salt or ester thereof and useful ranges may be selected between any of these values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%).

In one embodiment the composition comprises at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight VA or a salt or ester thereof and useful ranges may be selected between any of these values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%).

In another embodiment the ratio of cis-9, trans-11 CLA or a salt or ester thereof to VA or a salt or ester thereof in a composition of the invention or in a composition delivered to a subject according to the invention is about 1:100 to about 100:1, about 1:10 to about 10:1, about 1:5 to about 5:1, about 1:2 to about 2:1, preferably about 2:3 or about 3:2. In still another embodiment the ratio of cis-9, trans-11 CLA or a salt or ester thereof to VA or a salt or ester thereof in a composition of the invention or in a composition delivered to a subject according to the invention is about 0.5:9.5 to about 9.5:0.5, or about 0.5:9.5 to about 3:7, or about 1:12 to about 1:6

In one embodiment the composition comprises about 0.001 grams to about 19 grams of cis-9, trans-11 CLA or a salt or ester thereof and 0.001 grams to about 19 grams of VA or a salt or ester thereof. In another embodiment where the composition is a bulk consumer product, the composition may comprise up to about 130 grams of cis-9, trans-11 CLA or a salt or ester thereof and about 550 grams of VA or a salt or ester thereof.

In one embodiment the composition comprises at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of cis-9, trans-11 CLA or a salt or ester thereof and useful ranges may be selected between any of these values (for example, from about 0.01 to about 1 grams, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams).

In one embodiment the composition comprises at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of VA or a salt or ester thereof and useful ranges may be selected between any of these values (for example, from about 0.01 to about 1 grams, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams).

In one embodiment the composition further comprises, consists essentially of or consists of about 0.1, 0.5, 1, 5, 10, 15, 20, 25, 30, 35, 40, 45 or 50% by weight of fresh, recombined or powdered whole milk or a milk derivative and useful ranges may be selected between any of these values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, and from about 45 to about 50%). The milk derivative is preferably selected from recombined, powdered or fresh skim milk, reconstituted whole or skim milk powder, skim milk concentrate, skim milk retentate, concentrated milk, buttermilk, ultrafiltered milk retentate, milk protein concentrate (MPC), milk protein isolate (MPI), calcium depleted milk protein concentrate (MPC), low fat milk, low fat milk protein concentrate (MPC), casein, caseinate, milk fat, anhydrous milk fat (AMF), colostrum, a colostrum fraction, colostrum protein concentrate (CPC), colostrum whey, an immunoglobulin fraction from colostrum, whey, whey protein isolate (WPI), whey protein concentrate (WPC), sweet whey, lactic acid whey, mineral acid whey, reconstituted whey powder, a composition derived from any milk or colostrum processing stream, a composition derived from the retentate or permeate obtained by ultrafiltration or microfiltration of any milk or colostrum processing stream, or a composition derived from the breakthrough or adsorbed fraction obtained by chromatographic (including but not limited to ion and gel permeation chromatography) separation of any milk or colostrum processing stream.

In one embodiment the composition further comprises a pharmaceutically acceptable carrier. In one embodiment the composition is in the form of a tablet, a caplet, a pill, a hard or soft capsule or a lozenge. In one embodiment the composition is in the form of a cachet, a dispensable powder, granules, a suspension, an elixir, a liquid, or any other form that can be added to food or drink, including for example water or fruit juice.

In one embodiment the composition comprises, consists essentially of or consists of cis-9, trans-11 CLA or a salt or ester thereof, VA or a salt or ester thereof and anti-inflammatory food component. In another embodiment the composition comprises, consists essentially of or consists of cis-9, trans-11 CLA or a salt or ester thereof, VA or a salt or ester thereof and an anti-inflammatory milk component.

In one embodiment the cis-9, trans-11 CLA or ester thereof is selected from cis-9, trans-11 CLA derived from a natural source (isolated from animal or plant sources, for example); synthetic cis-9, trans-1 CLA; cis-9, trans-11 CLA in free fatty acid form; cis-9, trans-11 CLA in esterified form; cis-9, trans-11 CLA bound to glycerol including in monoglyceride, diglyceride or triglyceride form; cis-9, trans-11 CLA bound to a phospholipid, with or without other fatty acids; or mixtures thereof.

In one embodiment the composition further comprises one or more constituents (such as antioxidants) which prevent or reduce degradation of the composition during storage or after administration.

In one embodiment the composition is or is formulated as a food, drink, food additive, drink additive, dietary supplement, nutritional product, medical food, nutraceutical, medicament or pharmaceutical. Preferably, the composition is or is formulated as a powder, liquid, food bar, spread, sauce, ointment, tablet or capsule. In one embodiment, the composition is a milk powder, milk drink, yoghurt, yoghurt powder, yoghurt drink, butter or cheese.

In one embodiment the composition is formulated for oral, nasal, topical or parenteral (including subcutaneous, intramuscular and intravenous) administration.

In one embodiment the composition is formulated for ingestion, inhalation or topical application. Where the composition is formulated for inhalation, preferably it is formulated as an inhalable powder, solution or aerosol. Where the composition is formulated for topical application, preferably it is formulated as an ointment, cream or lotion.

In one embodiment the composition is formulated for separate, simultaneous or sequential administration of cis-9, trans-11 CLA or a salt or ester thereof and VA or a salt or ester thereof. In one embodiment separate compositions are formulated for separate, simultaneous or sequential administration of cis-9, trans-11 CLA or a salt or ester thereof and VA or a salt or ester thereof.

In one embodiment the cis-9, trans-11 CLA or a salt or ester thereof, VA or a salt or ester thereof and an agent selected from therapeutic agents including but not limited to bronchodilators, anticholinergic agents and anti-inflammatory agents are administered separately, simultaneously or sequentially.

In one embodiment the condition is an atopic condition. In another embodiment the condition is an allergy. In yet another embodiment the condition is an eosinophilia. In still another embodiment the condition is a Th2 mediated condition.

In one embodiment the condition is selected from allergic rhinitis, hay fever, atopic rhinoconjunctivitis, urticaria, asthma and atopic eczema.

In one embodiment the condition is selected from contact dermatitis, eczema (also referred to as allergic dermatitis or atopic dermatitis), hives (urticaria), allergic conjunctivitis, hay fever, allergic rhinitis, airborne allergies including tree (e.g. birch pollen), weed (e.g. ragweed), and grass pollen allergies, latex allergies, food allergies (e.g. peanut, shellfish, milk protein), drug allergies (e.g. to penicillin), insect sting allergies (e.g. honeybee allergies, wasp allergies, hornet allergies, yellow jacket allergies, fire ant allergies), mold allergies (e.g. to alternaria, cladosporium, aspergillus, penicillium, helminthosporium, epicoccum, fusarium, mucor, rhizopus, and aureobasidium), dust mite allergies, animal allergies (e.g. household pets such as cats and dogs), allergic bronchopulmonary aspergillosis, occupational asthma, and episodic angioedema with eosinophilia.

In one embodiment the condition is selected from airway, lung, blood and skin eosinophilia. In another embodiment, the eosinophilia is selected from eosinophilic ascites, eosinophilic cellulitis, eosinophilic fascitis, eosinophilic gastroenteritis, coeliac disease, allergic colitis, eosinophilic esophagitis, eosinophilic pancreatitis, eosinophilic pneumonias, bronchiectasis, eosinophilic synovitis, nasal eosinophilia, tropical pulmonary eosinophilia, Churg Strauss syndrome, pulmonary eosinophilia, idiopathic hyper-eosinophilic syndrome, inflammatory bowel disease, eosinophilic cholangitis, eosinophilic leukaemia and other eosinophilic cancers, familial (hereditary eosinophilia), eosinophilic granuloma, sarcoidosis, eosinophilia-myalgia syndrome, cystic fibrosis, nasal polyposis, eosinophil meningitis, Wegener's granulomatosis, polyarteritis nodosa, rheumatoid arthritis, pemphigus vulgaris, bullous pemphigoid, dermatitis herpetiformis, erythema multiforme, eosinophilic cellulites, parasitic infections (Ascaris Toxocara canis, Filariasis, Anchylostomiasis, Trichinosis, Strongvloidiasis, Fascioliasis, Schistosomiasis).

In one embodiment the condition is selected from Th2 mediated asthma, allergies, eczema, microbial or parasite infection, and autoimmune diseases including ulcerative colitis.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### BRIEF DESCRIPTION OF THE DRAWINGS

FIG. 1 is a graph showing that feeding a diet containing a combination of VA and cis-9, trans-11 CLA inhibits airway inflammation in a mouse model of asthma. Mice fed the AIN93G diet, or the same diet in which the soybean oil was partially substituted with VA, cis-9, trans-11 CLA, or a combination of identical amounts of each of the latter supplements were immunized and challenged intranasally with OVA. A BAL was performed on all mice six days after the OVA challenge. The mean number of total BAL cells, monocytes/macrophages, lymphocytes, and eosinophils (±SEM) is shown (n=5 to 6 mice per group). The * denotes statistical significance from control.

FIG. 2 is a graph showing the histopathology scores determined from inspection of alcian blue-PAS stained paraffin embedded sections of the left lung of each animal. Lung inflammation, perivascular/peribronchiolar infiltrates, airway epithelial hypertrophy, goblet-cell hyperplasia, constriction of bronchioles, and beneficial presence of phagocytic macrophages were graded on a scale of 0 (no change) to 4 (marked change). Each animal received an overall histopathology score based on summation of individual scores for each criteria. Sections were inspected for mucin hypersecretion, which was also graded on a scale of 0 (no change) to 4 (marked change), with each animal receiving a mucus index. All slides were scored in a blinded fashion (blinded to diet treatment/group assignment), and scores were presented as the mean A SEM of 5-6 animals/group. The * denotes statistical significance from control.

FIG. 3 is a graph showing IL-5 levels in bronchial fluid after allergen challenge as determined by ELISA. Data are expressed as mean±SEM, n=5 to 6 mice. The * denotes statistical significance from control.

### DETAILED DESCRIPTION OF THE INVENTION

### 1. Definitions

The term “comprising” as used in this specification and claims means “consisting at least in part of”. When interpreting statements in this specification and claims that include that term, the features, prefaced by that term in each statement, all need to be present but other features can also be present. Related terms such as “comprise” and “comprised” are to be interpreted in the same manner.

An “effective amount” is the amount required to confer therapeutic effect. The interrelationship of dosages for animals and humans (based on milligrams per meter squared of body surface) is described by Freireich, et al. (1966). Body surface area can be approximately determined from height and weight of the subject. See, e.g., Scientific Tables, Geigy Pharmaceuticals, Ardley, N.Y., 1970, 537. Effective doses also vary, as recognized by those skilled in the art, dependent on route of administration, carrier usage, and the like.

The terms “enriched with cis-9, trans-11 CLA and VA” and “enriched with a composition consisting of cis-9, trans-11 CLA and VA” are intended to mean that cis-9, trans-11 CLA or a salt or ester thereof and VA or a salt or ester thereof or a composition essentially consisting of, or consisting of cis-9, trans-11 CLA or a salt or ester thereof and VA or a salt or ester thereof has been added to a food, drink, food additive, drink additive, dietary supplement, nutritional product, medical food or nutraceutical composition so that it has a higher concentration of cis-9, trans-11 CLA or a salt or ester thereof and VA or a salt or ester thereof than it did before the cis-9, trans-11 CLA or a salt or ester thereof and VA or a salt or ester thereof were added. Preferably a composition is enriched by 5, 10, 15, 20, 25, 30, 35, 40, 45, 50, 55, 60, 65, 70, 75, 80, 85, 90, 95, 99, 99.5, 99.8 or 99.9% by weight with cis-9, trans-11 CLA or a salt or ester thereof and VA or a salt or ester thereof or a composition consisting of cis-9, trans-11 CLA or a salt or ester thereof and VA or a salt or ester thereof compared to the total final weight of the combined composition.

The term “pharmaceutically acceptable carrier” is intended to refer to a carrier including but not limited to an excipient, diluent, auxiliary or combination thereof that can be administered to a subject as a component of a composition of the invention that does not reduce the activity of the composition and is not toxic when administered in doses sufficient to deliver an effective amount of cis-9, trans-11 CLA or a salt or ester thereof and VA or a salt or ester thereof. The formulations can be administered orally, nasally, topically or parenterally (including intramuscularly, intraperitoneally, subcutaneously and intravenously).

A “subject” in accordance with the invention is an animal, preferably a mammal, more preferably a mammalian companion animal or human. Preferred companion animals include cats, dogs and horses.

The term “steroid sparing” is intended to mean that the dose of steroidal medication administered to a subject is able to be reduced to a level below that administered before the subject began taking a composition of the present invention. Preferably the daily or weekly or monthly dose is able to be reduced by at least 10, 15, 20, 25, 30, 35, 40, 45, 50, 55, 60, 65, 70, 75, 80, 85, 90, 95 or 99%.

The term “treat” and its derivatives should be interpreted in their broadest possible context. The term should not be taken to imply that a subject is treated until total recovery. Accordingly, “treat” broadly includes amelioration and/or prevention of the onset of the symptoms or severity of a particular condition; for example reduction in leukocyte infiltration or eosinophilia, lesions, or preventing or otherwise reducing the risk of developing an allergic response, or disease symptom. The term “treat” also broadly includes the maintenance of good respiratory health for sensitive individuals and building stamina for disease prevention.

### 2. A Combination of Cis-9, Trans-11 CLA and VA is Useful to Treat Asthma

A combination of cis-9, trans-11 CLA and VA was able to attenuate many of the symptoms of asthma including lung inflammation (including eosinophilia), airway epithelial hypertrophy, goblet-cell hyperplasia, leukocyte infiltration, airway remodelling, bronchoconstriction (constriction of bronchioles) and mucus hypersecretion. This combination has efficacy in maintaining or restoring lung health, symptomatic relief of asthma or other allergic conditions and to reduce the expression of symptoms. The combination of VA and cis-9, trans-11 CLA significantly inhibited mucus hypersecretion, whereas significance was not achieved with the cis-9, trans-11 CLA alone or with VA alone.

Santora et al. (2000) reported that ˜12% of the VA consumed by mice during a two week feeding period was retained as cis-9, trans-11 CLA in its triglyceride form. For comparison, the average conversion rate in humans is ˜19% (Turpeinen, et al., 2002). In the Examples below mice were fed 2 g of cis-9, trans-11 CLA and 3 g of VA per 1.4 Kg of diet.

Without wishing to be bound by theory, given the findings of Santora et al. (2000), the VA fed to mice could be expected to elevate the level of dietary cis-9, trans-11 CLA by just 18% to 2.36 g. An 18% increase in the level of cis-9, trans-11 CLA does not explain the effects seen with the combination diet.

VA and cis-9, trans-1 CLA fed individually had no significant effect on lung pathology and both increased leukocyte infiltration into the airway lumen. The combination of VA and cis-9, trans-11 CLA significantly reduced leukocyte infiltration, particularly lymphocyte and eosinophil infiltration.

The results described below indicate that VA acid and cis-9, trans-11 CLA synergize to inhibit both allergen-specific remodelling and inflammation of the lung.

Diets containing a combination of VA and cis-9, trans-1 CLA may have utility in preventing and/or treating the symptoms of asthma, and related disorders.

It is believe that diets containing a combination of VA and cis-9, trans-11 CLA may also have utility in preventing and/or treating the symptoms of dermatitis, and related disorders.

### 3. Sources of Cis-9, Trans-11 CLA and VA

The cis-9, trans-11 CLA or a salt or ester thereof and VA or a salt or ester thereof may be synthetic, derived from a natural source, or mixtures thereof. Natural sources of cis-9, trans-11 CLA are described by Chin et al (1992) and include animal, bacterial and plant sources. Linoleic acid may be converted to CLA by bacterial fermentation with Clostridium sporogenes, Clostridium bifermentans, Clostridium sordellii and Bacteroides sp, for example (Verhulst, et al., 1985). Other useful organisms for bacterial fermentation include Butyrivibrio fibrisolvens, Eubacterium lentum, Propionibacterium freudenreichi, Lactobacillus acidophilus, Lactobacillus reuteri, Megasphaera elsdenii, and Bifidobacterium breve. Linoleic acid may be converted to CLA and VA by bacterial fermentation with Butyrivibrio fibrisolvens (Fukuda, et al., 2005).

In one embodiment where the cis-9, trans-11 CLA or salt or ester thereof and/or VA or salt or ester thereof are synthetic, the CLA and/or VA may be chemically modified to improve potency, stability, transport and half-life.

In one embodiment, the cis-9, trans-11 CLA or the VA or both may be included in a composition of the invention in free fatty acid form. In another embodiment the cis-9, trans-11 CLA or the VA or both may be in an esterified form, including but not limited to alkyl esters (including but not limited to methyl, ethyl, propyl, isopropyl, butyl, sec-butyl, isobutyl, tert-butyl, pentyl, hexyl, and heptyl esters). In another embodiment the cis-9, trans-1 CLA or the VA or both may be in a salt form, including but not limited to sodium salts and zinc salts. In a further embodiment, one or more cis-9, trans-11 CLA or VA molecules or molecules of both may be bound to the same or separate polyol such as glycerol or sphingosine, with or without other fatty acids, to form mono-, di- or tri-glycerides for example. In still another embodiment the cis-9, trans-11 CLA or the VA or both may be bound to the same or separate phospholipid (including but not limited to phosphatidylethanolamines, phosphatidylinositols, phosphatidylserines, phosphatidylcholines and sphingomyelins) or ceramide (including but not limited to glucoceramides and lactoceramides), with or without other fatty acids. In yet another embodiment, mixtures of these forms of cis-9, trans-11 CLA and VA may be included within a composition of the invention.

Sunflower and safflower seed oils, containing approximately 65% and 76% linoleic acid respectively, are currently used as raw material for CLA production. Optimal conditions used in commercial scale production results in approximately equal amounts of the isomers cis-9, trans-11 and trans-10, cis-12. A safflower based product can thus contain approximately 36% each of cis-9, trans-11 and trans-10, cis-12 isomers. Minor peaks include the cis, cis and trans, trans isomers of 9,11 and 10,12 CLA, each around 0.5 to 1%. Traces of cis-11, trans-13 (which is formed from heating the trans-10, cis-12 isomer) and trans-8, cis-10 (from heating of the cis-9, trans-11 isomer) may also be present.

A composition for use according to the invention may optionally further comprise at least one antioxidant or other agent able to prevent degradation of the cis-9, trans-11 CLA or VA or the salts or esters thereof.

VA may be natural (including VA produced by bacterial fermentation or isolated from natural sources such as milk) or synthetic.

Alternately, VA, cis-9, trans-1 CLA, their mixtures, and/or mixtures of the metabolic intermediates that lead to the formation of cis-9, trans-11 CLA and VA may be obtained from any microbial fermentation process that uses unsaturated fatty acids as feedstocks of the process and rumen bacteria for the fermentation.

### 4. Compositions Useful According to the Invention

A composition useful herein may be formulated as a food, drink, food additive, drink additive, dietary supplement, nutritional product, medical food, nutraceutical, medicament or pharmaceutical. Preferably, a composition of the invention is formulated as a powder, liquid, food bar, spread, sauce, ointment, tablet or capsule. Suitable foods and drinks include dairy and non-dairy foods and drinks. In one embodiment, the composition is a milk powder, milk drink, yoghurt, yoghurt powder, yoghurt drink, butter or cheese. Appropriate formulations may be prepared by an art skilled worker with regard to that skill and the teaching of this specification.

The compositions useful herein may be formulated to allow for administration to a subject by any chosen route, including but not limited to oral, nasal, topical or parenteral (including subcutaneous, intramuscular and intravenous) administration.

Thus, a pharmaceutical composition of the invention may be formulated with an appropriate pharmaceutically acceptable carrier (including excipients and diluents) selected with regard to the intended route of administration and standard pharmaceutical practice. For example, a composition of the invention can be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

The compositions can also be administered by inhalation (orally or intranasally), and are conveniently delivered in the form of a dry powder inhaler or an aerosol spray presentation from a pressurised container, pump, spray, atomiser or nebuliser, with or without the use of a suitable propellant as known in the art.

In one preferred embodiment, a composition for use according to the invention is formulated for ingestion, inhalation or topical application.

The compositions useful herein may be used alone or in combination with one or more other therapeutic agents. The therapeutic agent may be a food, drink, food additive, drink additive, food component, drink component, dietary supplement, nutritional product, medical food, nutraceutical, medicament or pharmaceutical. The therapeutic agent is preferably effective to attenuate one or more of the symptoms of asthma, maintain or restore lung health, aid in symptomatic relief of asthma or other allergic conditions or to reduce the expression of allergic symptoms. Alternatively or additionally, the therapeutic agent is preferably effective to attenuate one or more of the symptoms of dermatitis, maintain or restore skin health, or aid in symptomatic relief of dermatitis.

In use, optionally in combination with another therapeutic agent, the administration of cis-9, trans-11 CLA or a salt or ester thereof, VA or a salt or ester thereof and the optional therapeutic agent may be simultaneous or sequential. Simultaneous administration includes the administration of a single dosage form that comprises all components and the administration of cis-9, trans-11 CLA or a salt or ester thereof, VA or a salt or ester thereof and the optional therapeutic agent in separate dosage forms at substantially the same time. Sequential administration includes the administration of cis-9, trans-11 CLA or a salt or ester thereof, VA or a salt or ester thereof and the optional therapeutic agent according to different schedules, preferably so that there is an overlap in the periods during which the cis-9, trans-11 CLA or a salt or ester thereof, VA or a salt or ester thereof and the optional therapeutic agent are provided.

Suitable agents with which the compositions of the invention can be co-administered include bronchodilators (e.g. beta-2 agonists), anticholinergic agents (e.g. antimuscarinic agents and antinicotinic agents), or anti-inflammatory agents (e.g. inhaled steroids, intranasal steroids, steroid creams and ointments, oral steroids and leukotriene antagonists and 5-lipoxygenase inhibitors), and other suitable agents known in the art.

In one embodiment a composition of the invention may further comprise or be administered with one or more anti-inflammatory milk components including but not limited to vitamin D, a casein hydrolysate, one or more casein peptides known to be immunosuppressive, taurine, beta-lactoglobulin and fragments thereof, TGF-beta, glycomacropeptide or a fraction thereof, osteopontin and fragments thereof, omega3 fatty acids, butyrophilin, a growth factor-enriched fraction from milk whey, and phytanic acid. Preferably the composition is a food, drink, food additive, drink additive, dietary supplement, nutritional product, medical food or nutraceutical. Milk fractions enriched for these components may also be employed.

In one embodiment a composition of the invention may further comprise or be administered with one or more anti-inflammatory food components including but not limited to vitamin E; vitamin C; Lyprinol™; bromelain; a bioflavonoid mixture extracted from Pinus maritime (pine bark) such as Pycnogenol™; garlic; extracts of Ginkgo biloba leaves; Ephedra (ma-huang); a combination of three Chinese herbal extracts (Ling-Zhi (Ganoderma lucidum), Ku-Shen (Radix Sophora flavescentis) and Gan-Cao (Radix Glycyrrhiza uralensis)) known as ASHMI for “antiasthma herbal medicine intervention”; Oxy 17™ available from Progressive Health Nutraceuticals, Inc. (USA); extracts from the mushrooms Cordyceps sinensis, Ganoderma lucidium (Reishi), and Tremella fuciformis (Silver-Ear); perilla leaf extract; rosmarinic acid; flavonoids (such as luteolin, fisetin and apigenin); simple sugars (such as L-fucose and N-acetylneuraminic acid); methylsulfonylmethane; soy protein or genistein or both; quercetin; spirulina; forskolin; and mixtures thereof. Preferably the composition is a food, drink, food additive, drink additive, dietary supplement, nutritional product, medical food or nutraceutical.

In one embodiment, a pharmaceutical composition further comprises, or is formulated for administration (simultaneous or sequential) with, an agent selected from bronchodilators, corticosteroids, long-acting beta agonists, leukotriene modifiers and other suitable agents known in the art. In one embodiment bronchodilators include but are not limited to beta-2 agonists; anticholinergic agents include but are not limited to antimuscarinic agents and antinicotinic agents; and anti-inflammatory agents include but are not limited to inhaled steroids, intranasal steroids, steroid creams and ointments, oral steroids, leukotriene receptor antagonists, leukotriene antagonists and 5-lipoxygenase inhibitors.

In one embodiment a composition of the invention may further comprise or be administered with one or more of inhaled or oral steroids (including but not limited to beclomethasone (Beclovent®, Vanceril®, Becloforte®), budesonide (Pulmicort®), flunisolide (Bronalide®), and fluticasone (Flovent®)); corticosteroids (including but not limited to prednisone); Nedocromil; ketotifen; beta-2 agonists (including but not limited to salbutamol (Ventolin®, Apo-Salvent®, Novo Salmol®), fenoterol (Berotec®), terbutaline (Bricanyl®), and pirbuterol (Maxair®); theophylline; leukotriene antagonists; leukotriene receptor antagonists (including but not limited to zafirlukast (Accolate®), and montelukast (Singulair®); 5-lipoxygenase inhibitors; anticholinergics (eg Atrovent®); Zileuton; Zafirlukast; macrolide azalide antimicrobial agents; ketolide antimicrobial agents; 5,8,11,14-eicosatetraynoic acid (ETYA); lipoxins and lipoxin and LXA4 analogues; triterpenoids from Vochysia pacifica; cromolyn; antibodies against IgE (eg Omalizumab), IL-4 and IL-5; antibodies against cell adhesion molecules and chemokines; anti-inflammatory cytokines such as IL-10; DNA-based methods of immunization (CpG DNA, CpG DNA conjugated to a protein allergen, and plasmid DNA); magnesium sulphate; blockers of IL-5-IL-5 receptor interaction; blockers of eotaxin-CCR3 interaction; blockers of the binding of IgE to mast cells; inhibitors of phosphodiesterase (PDE-4) or p38 kinase; and antihistamines.

It should be understood that the additional therapeutic agents listed above (both food based and pharmaceutical agents) may also be employed in a method of the invention where they are administered separately, simultaneously or sequentially with cis-9, trans-11 CLA or a salt or ester thereof and VA or a salt or ester thereof or a composition comprising, consisting essentially of or consisting of cis-9, trans-11 CLA or a salt or ester thereof and VA or a salt or ester thereof.

As will be appreciated, the dose of the composition administered, the period of administration, and the general administration regime may differ between subjects depending on such variables as the severity of symptoms of a subject, the type of disorder to be treated, the mode of administration chosen, and the age, sex and/or general health of a subject. However, by way of general example, the inventors contemplate administration of from about 1 mg to about 1000 mg per kg body weight of a composition of the invention is administered per day, preferably about 50 to about 500 mg per kg per day. In one embodiment, the inventors contemplate administration of from about 0.05 mg to about 250 mg per kg body weight of a pharmaceutical composition according to the invention.

It should be appreciated that administration may include a single daily dose or administration of a number of discrete divided doses as may be appropriate.

Various aspects of the invention will now be illustrated in non-limiting ways by reference to the following examples.

### EXAMPLES

### Mice

Eight to nine week old male and female C57BL/6 mice (University of Auckland, New Zealand) were kept on an ovalbumin (OVA)-free normal mouse chow diet from weaning up until they were assigned to a particular diet (control or experimental diet). Each diet group (n=6) contained an equal number of male and female mice. Throughout the study period mice were kept in an air-conditioned room with controlled humidity, temperature, and a 12h light:dark cycle.

### Diets

Healthy control mice were maintained on an unmodified AIN-93G diet. Soybean oil (which contains no CLA) was the dietary fat source in the AIN-93G diet. The final fat content of all treatment diets used in the Examples was maintained at the same level as the fat content of the control AIN-93G diet by reducing the soybean oil content of the treatment or control diets as required. Fresh diet was provided biweekly, and mice had free access to food and water throughout the study.

### Sensitization and Allergen Exposure Protocol

Body weights were determined, and blood samples collected via the tail vein, prior to assignment of mice to particular diets. Blood triglyceride levels were measured using an Accutrend GCT meter (Roche Diagnostics, Germany). After two weeks on an assigned diet mice were immunized with two intraperitoneal (i.p.) injections of 20 μg of OVA (chicken egg albumin grade V; Sigma Chemical Co., St Louis, Mo.) complexed with 2 mg of Imject Alum (Al(OH)₃/Mg(OH)₂; Pierce Rockford IL) in a total volume of 100 μl of PBS on days 0 and 14. Two weeks after the 2nd injection mice were anaesthetized by i.p. injection of a mixture of ketamine and xylazine (Phoenix, Auckland, New Zealand), and challenged intranasally with 100 μg of OVA in 50 μl of PBS. Body weights and final blood triglyceride levels were measured. Blood, bronchoalveolar lavage (BAL) fluid, lung, liver and spleen tissue samples were collected 6 days after the intranasal challenge. Blood was collected by cardiac puncture after deeply anaesthetizing mice by i.p. injection of a mixture of ketamine and xylazine. Serum was separated from blood samples, and stored at −80° C.

Assessment of Inflammatory Cell Infiltration into the Lung

Bronchoalveolar lavage was performed immediately after euthanasia by flushing 1 ml of PBS containing 1% heat inactivated fetal calf serum (lavage buffer) thrice through the lung and airways of mice via the cannulated trachea. The recovered BAL was pooled for each animal, centrifuged at 1,500 rpm at 4° C., and the supernatant stored at −80° C. The cell pellets were resuspended in 1 ml of lavage buffer, and total cell numbers were counted using a hemocytometer. BAL cells were centrifuged onto poly-L-lysine-coated glass slides using a cytospin, and stained with Diff-Quik stain (Dade Behring Inc. USA). Differential cellular counts were made by counting ≧300 cells under light microscopy (Nikon E200 microscope), using standard morphological criteria.

### Histochemistry

Following BAL, the left lobes of lungs were fixed in 4% paraformaldehyde in 0.1 M PBS (pH 7.4) overnight and paraffin embedded for histopathological analysis. The right lobes of lungs were kept frozen at −80° C. until further analysis. Sections were stained with Alcian Blue-Periodic Acid Schiff for the detection of acid and neutral mucins and identification of goblet cells and leukocytes.

### ELISA

The levels of IL-5 and eotaxin in the BAL fluid were quantitated by ELISA using a Quantikine mouse IL-5 ELISA kit and a mouse eotaxin Quantikine ELISA kit (R&D Systems, MN), respectively, according to the manufacturers' instructions. The detection limits were 3 pg/ml for eotaxin and 7 pg/ml for IL-5.

### Statistical Analysis

Data are expressed as the mean±SEM, and statistical significance was determined by the Student's t test. A value of P<0.05 was taken as significant.

### Example 1

### VA and Cis-9, Trans-11 CLA Diminish Leukocyte Infiltration in a Mouse Model of Asthma

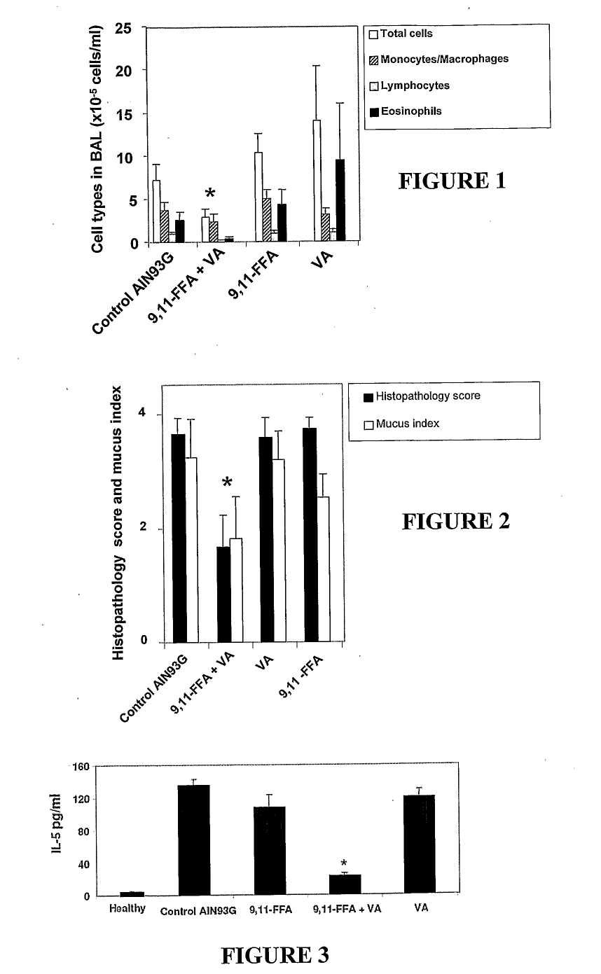

VA (99%) (Nu-Chek, Inc., USA) and cis-9, trans-11 CLA (90%) (Larodan Fine Chemicals AB, Sweden) were tested for their ability to attenuate the symptoms of OVA-induced asthma. Mice were fed a control AIN93G diet (contains no CLA isomer or VA) and diets containing either 0.14% of the cis-9, trans-11 CLA isomer [˜2% (w/w) of the fat content] in the free fatty acid form, 0.21% VA [˜3% (w/w) of the fat content], or both 0.14% of the cis-9, trans-11 CLA and 0.21% VA. VA alone significantly increased (by 59%, P<0.05) the levels of blood triglyceride (data not shown). For all the diets there was no significant change in the percent liver/body or spleen/body weight ratios (data not shown). Neither the diet supplemented with the cis-9, trans-11 isomer, nor the diet supplemented with VA, suppressed allergen-induced accumulation of leukocytes into the lung compared to the control diet. Rather, the latter diets increased the total bronchiolar lavage (BAL) cell counts on average by 43% (P<0.05) and 94% (P=0.06), respectively, and increased the accumulation of eosinophils by 69% (P=0.08) and 270% (P=0.07), respectively (FIG. 1), though the latter increases did not reach significance due to the large standard deviation. The increase in leukocyte infiltration from feeding the cis-9, trans-11 isomer was partly due to an increase (35%, P=0.05) in monocytes/macrophages, which are generally protective. Neither supplement had a significant effect on infiltration by lymphocytes. In contrast, the diet containing the combination of the cis-9, trans-11 CLA isomer and VA on average reduced the total BAL cell counts by 60% (P<0.01) compared to those obtained from mice fed the control diet (FIG. 1). The combination of the two supplements on average suppressed the accumulation of eosinophils by 86% (P<0.01) compared to the control diet (FIG. 1). The decrease in eosinophils was accompanied by a 38% (P<0.05) reduction in the numbers of monocytes/macrophages compared to the numbers of monocytes/macrophages in the BAL of mice fed the control diet. There was a 79% (P=0.001) reduction in the numbers of lymphocytes, compared to mice fed the control diet. Neutrophil numbers in the BAL were very low irrespective of the type of diet, and did not increase significantly following allergen challenge, and hence were not further analysed. Thus, the combination of VA and cis-9, trans-11 CLA isomer suppresses leukocyte, in particular eosinophil, infiltration into the lumen of the bronchial airways in response to allergen.

### Example 2

### VA and Cis-9, Trans-11 CLA Inhibit Airway Changes

Asthmatic animals were fed either the control AIN93G diet, the VA diet, or the cis-9, trans-11 CLA diet, as discussed above. In addition to massive peribronchial and perivascular infiltrates of leukocytes, there was marked epithelial cell hypertrophy, and goblet cell metaplasia. Furthermore, the alcian blue-periodic acid Schiff double staining method showed that the airway epithelial content of neutral mucopolysaccharides stained “red” with Schiff's reagent increased dramatically in response to allergen challenge. In marked contrast, similar changes to the lungs of allergen challenged mice fed the diet containing the combination of VA and cis-9, trans-11 CLA were significantly reduced. Changes to the lung were scored for perivascular/peribronchiolar infiltrates, beneficial presence of phagocytic macrophages, airway epithelial hypertrophy, goblet-cell hyperplasia, and constriction of bronchioles to give an overall histopathology score (FIG. 2). They were also scored for mucus hypersecretion to give a mucus index (FIG. 2). The histopathology score and mucus index were reduced in mice fed the combination of VA and cis-9, trans-1 CLA by 55% (P<0.001) and 44% (P<0.05), respectively, compared to the scores for mice fed the control diet. The bronchial airways of mice fed the combination diet appeared to be less constricted than those of mice fed either the control diet, the VA diet, or the cis-9, trans-11 CLA diet, and considerably less eosinophils remained in the lung tissue. Occasionally, macrophages could be detected that had engulfed large numbers of clusters of free eosinophil granules (cfegs) in common with the situation in the BAL.

### Example 3

### VA and Cis-9, Trans-11 CLA Inhibit IL-5 Expression in the Bronchial Airway

IL-5 and eotaxin produced by a variety of different cell types in the sensitized lung play key roles in asthma by controlling the development and release of eosinophils from the bone marrow, and their accumulation, activation and survival in the lung (Walsh et al., 2005; Shinagawa et al., 2003). Challenge with allergen led to marked increases in the levels of IL-5 and eotaxin (data not shown) in the BAL fluid of control mice fed the AIN93G diet (FIG. 3). The diet containing a combination of VA and cis-9, trans-11 CLA markedly reduced the expression of IL-5 in the BAL fluid by 83% (P=0.0005), compared to that of control mice fed the AIN-93G diet. In contrast, diets containing either VA or cis-9, trans-11 CLA only slightly reduced IL-5 levels by 11 and 21%, respectively. The level of eotaxin was similarly reduced by the diet containing a combination of VA and cis-9, trans-11 CLA, but did not reach significance, whereas the single bioactives were not effective (data not shown).

### INDUSTRIAL APPLICATION

The present invention has utility in treating or preventing conditions associated with one or more of leukocyte infiltration, eosinophilia, airway remodelling, bronchoconstriction, mucus hypersecretion, and lung and skin inflammation. The described compositions may be employed as foods, drinks, food additives, drink additives, dietary supplements, nutritional products, medical foods, nutraceuticals, medicaments or pharmaceuticals. The described compositions and methods of the invention may be employed to treat or prevent one or more of the conditions discussed above.

Those persons skilled in the art will understand that the above description is provided by way of illustration only and that the invention is not limited thereto.

### REFERENCES

Awad A, Hermann T, Finsk C S, Horvath P J. 18:1 n7 Fatty acids inhibit growth and decrease inositol phosphatase release in HT-29 cells compared to n9 fatty acids. Cancer Lett. 91: 55-61, 1995.
Banni S, Angioni E, Murru E, Carta G, Melis M P, Bauman D, Dong Y, Ip C. Vaccenic acid feeding increases tissue levels of conjugated linoleic acid and suppresses development of premalignant lesions in rat mammary gland. Nutr Cancer. 41:91-7, 2001.
Bergamo P, Luongo D, Maurano F, Rossi M. Butterfat fatty acids differentially regulate growth and differentiation in Jurkat T-cells. J Cell Biochem. 96:349-60, 2005.
Boguniewicz M, Leung D Y. Atopic dermatitis. J Allergy Clin Immunol. 2006; 117(2 Suppl Mini-Primer):S475-80.
Bolte G, Frye C, Hoelscher B, Meyer I, Wjst M, Heinrich J. Margarine consumption and allergy in children. Am. J. Respir. Crit. Care Med. 163: 277-279, 2001.
Chin, S.F., Liu, W., Storkson, J. M., Ha, Y. L. and Pariza, M. W. Dietary sources of conjugated dienoic isomers of linoleic acid, a newly recognised class of anticarcinogens, Journal of Food composition and Analysis, 5, 185-197, 1992.
Christodoulopoulos P, Cameron L, Durham S, Hamid, Q. Molecular pathology of allergic disease. II. Upper airway disease. J. Allergy Clin. Immunol. 105: 211-223, 2000.
Clement L, Poirier H, Niot I, Bocher V, Guerre-Millo M, Krief S, Staels B, Besnard P. Dietary trans-10,cis-12 conjugated linoleic acid induces hyperinsulinemia and fatty liver in the mouse. J. Lipid Res. 43: 1400-1409, 2002.
Corl B A, Barbano D M, Bauman D E, Ip C. cis-9, trans-11 CLA derived endogenously from trans-11 18:1 reduces cancer risk in rats. J. Nutr. 133:2893-900, 2003.
Dunder T, Kuikka L, Turtinen J, Rasanen L, Uhari M. Diet, serum fatty acids and atopic diseases in childhood. Allergy 56: 425-428, 2001.
Emelyanov A, Fedoseev G, Krasnoschekova O, Abulimity A, Trendeleva T, Barnes P J. Treatment of asthma with lipid extract of New Zealand green-lipped mussel: a randomised clinical trial. Eur. Respir. J. 20: 596-600, 2002.
Erjefalt J S, Greiff L, Andersson M, Matsson E, Petersen H, Linden M, Ansari T, Jeffery P K, Persson CGA. Allergen-induced eosinophil cytolysis is a primary mechanism for granule protein release in human upper airways. Am. J. Respir. Crit. Care Med. 160: 304-312, 1999.
Freireich E J, Gehan E A, Rall D P, Schmidt L H, Skipper H E (1966) Quantitative comparison of toxicity to anticancer agents in mouse, rat, hamster, dog, monkey and man. Cancer Chemother Rep 50: 219-244.
Frew A J, Plummeridge M J. Alternative agents in asthma. J. Allergy Clin. Immunol. 108: 3-10, 2001.
Fukuda S, Furuya H, Suzuki Y, Asanuma N, Hino T. A new strain of Butyrivibrio fibrisolvens that has high ability to isomerize linoleic acid to conjugated linoleic acid. J Gen Appl Microbiol. 2005 April; 51(2):105-13.
Gleich G J, Motojima S, Frigas E, Kephart G M, Fujisawa T, Kravis L P. The eosinophilic leukocyte and the pathology of fatal bronchial asthma: evidence for pathologic heterogeneity. J. Allergy Clin. Immunol. 80: 412-415, 1987.
Haby M M, Peat J K, Marks G B, Woolcock A J, Leeder S R. Asthma in preschool children: prevalence and risk factors. Thorax 56: 589-595, 2001.
Harfoot, C. G., and Hazlewood, G. P., “Lipid metabolism in the rumen” in P. N. Hobson (Ed.) “The Rumen Microbial Ecosystem” at pages 285 to 322, Elsevier Applied Science Publishers, London (1988).
Hodge L, Salome C M, Peat J K, Haby M M, Xuan W, Woolcock A J. Consumption of oily fish and childhood asthma risk. Med. J. Aust. 164: 137-40, 1996.
Kay A B. Allergy and allergic diseases. N. Eng. J. Med. 344: 30-37, 2001.
Kelley D S, Erickson K L. Modulation of body composition and immune function by conjugated linoleic acid in humans and animal models: benefits vs. risks. Lipids 38: 377-386, 2003.
Lamblin C, Gosset P, Salez, F, Vandezande L M, Perez T, Darras J, Janin A, Tonnel A B, Wallaert B. Eosinophilic airway inflammation in nasal polyposis. J. Allergy Clin. Immunol. 104: 85-92, 1999.
Lampen A, Leifheit M, Voss J, Nau H. Molecular and cellular effects of cis-9, trans-11-conjugated linoleic acid in enterocytes: effects on proliferation, differentiation, and gene expression. Biochim Biophys Acta. 1735: 30-40, 2005.
Leiferman K M. Eosinophils in atopic dermatitis. Allergy 44: 20-26, 1989.
Lopez-Garcia E, Schulze M B, Meigs J B, Manson J E, Rifai N, Stampfer M J, Willett W C, Hu F B. Consumption of trans fatty acids is related to plasma biomarkers of inflammation and endothelial dysfunction. J. Nutr. 135: 562-6, 2005.
Luna L G, editor. Manual of histologic staining methods of the Armed Forces Institute of Pathology. New York: McGraw-Hill; 1968.
Macdessi J S, Randell T L, Donaghue K C, Ambler G R, van Asperen P P, Mellis C M. Adrenal crises in children treated with high-dose inhaled corticosteroids for asthma. Med. J. Aust. 178: 214-216, 2003.
Milgrom H, Berger W, Nayak A, Gupta N, Pollard S, McAlary M, Taylor A F, Rohane P. Treatment of childhood asthma with anti-immunoglobulin E antibody (omalizumab). Pediatrics 108: E36, 2001.
Mozaffarian D, Rimm E B, King I B, Lawler R L, McDonald G B, Levy W C. Trans fatty acids and systemic inflammation in heart failure. Am J Clin Nutr. 80: 1521-5, 2004.
Nagakura T, Matsuda S, Shichijyo K, Sugimoto H, Hata K. Dietary supplementation with fish oil rich in ω-3 polyunsaturated fatty acids in children with bronchial asthma. Eur. Respir. J. 16: 861-865, 2000.
Niven A S, Argyros G. Alternate Treatments in Asthma. Chest 123:1254-1265, 2003.
Noli C, Carta G, Cordeddu L, Melis M P, Murru E, Banni S. Conjugated linoleic acid and black currant seed oil in the treatment of canine atopic dermatitis: A preliminary report. Vet J. 2006 Feb. 20; [Epub ahead of print].
O'Shea M, Bassaganya-Riera J, Mohede I C. Immunomodulatory properties of conjugated linoleic acid. Am J Clin Nutr. 79:1199 S-1206S, 2004.
Parodi P W. Health benefits of conjugated linoleic acid. Food Industry J. 3: 222-259, 2002.
Precht D, Molkentin J. Rapid analysis of isomers of transoctadecenoic acid in milk fat. Int Dairy J. 6:791-809, 1996.
Riserus U, Basu S, Jovinge S, Fredikson G N, Arnlov J, Vessby B. Supplementation with conjugated linoleic acid causes isomer-dependent oxidative stress and elevated C-reactive protein. A potential link to fatty acid-induced insulin resistance. Circulation 106: 1925-1929, 2002.
Santora J E, Palmquist D L, Roehrig K L. Trans-vaccenic acid is desaturated to conjugated linoleic acid in mice. J. Nutr. 130: 208-15, 2000.
Shinagawa K, Trifilieff A, Anderson G P. Involvement of CCR3-reactive chemokines in eosinophil survival. Int Arch Allergy Immunol 2003; 130:150-7.
Spector S L, Surette M E. Diet and asthma: has the role of dietary lipids been overlooked in the management of asthma Ann. Allergy Asthma Immunol. 90: 371-377, 2003.
Suissa S, Ernst P. Inhaled corticosteroids: Impact on asthma morbidity and mortality. J. Allergy Clin. Immunol. 107: 937-944, 2001.
Thomas P S, Geddes D M, Barnes P J. Pseudo-steroid resistant asthma. Thorax 54: 352-356, 1999.
Turpeinen A M, Mutanen M, Aro A, Salminen I, Basu S, Palmquist D L, Griinari J M. Bioconversion of vaccenic acid to conjugated linoleic acid in humans. Am J Clin Nutr. 76: 504-10, 2002.
Verhulst A, Semjen G, Meerts U, Janssen G, Parmentier G, Asselberghs S, van Hespen H & Eyssen H (1985). Biohydrogenation of linoleic acid by Clostridium sporogenes, Clostridium bifermentans, Clostridium sordellii and Bacteroides sp. FEMS Microbiology Ecology, 31, 255-259.
von Mutius E, Weiland S K, Fritzsch C, Duhme H, Keil U. Increasing prevalence of hayfever and atopy among children in Leipzig, East Germany. Lancet 351: 862-866, 1998.
Walsh G M, Al-Rabia M, Blaylock M G, Sexton D W, Duncan C J, Lawrie A. Control of eosinophil toxicity in the lung. Curr Drug Targets Inflamm Allergy 2005; 4:481-6.
Whigham L D, Cook E B, Stahl J L, Saban R, Bjorling D E, Pariza M W, Cook M E. CLA reduces antigen-induced histamine and PGE2 release from sensitized guinea pig tracheae. Am. J. Physiol. Regulatory Integrative Comp. Physiol. 280: R908-R912, 2001.
Whigham L D, Higbee A, Bjorling D E, Park Y, Pariza M W, Cook M E. Decreased antigen-induced eicosanoid release in conjugated linoleic acid-fed guinea pigs. Am. J. Physiol. Regulatory Integrative Comp. Physiol. 282: R1104-R1112, 2002.
Wijga A H, Smit H A, Kerkhof M, de Jongste J C, Gerritsen J, Neijens H J, Boshuizen H C, Brunekreef B. Association of consumption of products containing milk fat with reduced asthma risk in pre-school children: the PIAMA birth cohort study. Thorax 58: 567-572, 2003.
Woods R K, Thien F C, Abramson M J. Dietary marine fatty acids (fish oil) for asthma in adults and children. Cochrane Database Syst. Rev. CD001283, 2002
Wu D. Modulation of immune and inflammatory responses by dietary lipids. Curr Opin Lipidol. 15: 43-7, 2004.

1.-2. (canceled)

3. A composition comprising about 5% to about 30% by weight cis-9, trans-11 conjugated linoleic acid (CLA) or a salt or ester thereof and about 95% to about 70% by weight vaccenic acid (VA) or a salt or ester thereof.

4. A composition of claim 3 comprising cis-9, trans-11 CLA or a salt or ester thereof and VA or a salt or ester thereof in a ratio of about 0.5:9.5 to about 3:7 by weight, the composition comprising at least about 7% by weight cis-9, trans-11 CLA or a salt or ester thereof based on the weight of the composition.

5. A composition of claim 3 comprising cis-9, trans-11 CLA or a salt or ester thereof and VA or a salt or ester thereof in a ratio of about 1:12 to about 1:6 by weight, the composition comprising at least about 7% by weight cis-9, trans-11 CLA or a salt or ester thereof based on the weight of the composition.

6. A composition of claim 3, wherein the composition is a food, drink, food additive, drink additive, dietary supplement, nutritional product, medical food or nutraceutical.

7. A pharmaceutical composition comprising cis-9, trans-11 conjugated linoleic acid (CLA) or a salt or ester thereof, vaccenic acid (VA) or a salt or ester thereof and a pharmaceutically acceptable carrier.

8. A pharmaceutical composition of claim 7 comprising cis-9, trans-11 CLA or a salt or ester thereof, VA or a salt or ester thereof, a pharmaceutically acceptable carrier and one or more agents selected from bronchodilators, anticholinergic agents and anti-inflammatory agents.

9. A pharmaceutical composition of claim 7, wherein the composition comprises about 1% to about 99% by weight cis-9, trans-11 CLA or a salt or ester thereof and about 99% to about 1% by weight VA or a salt or ester thereof based on the combined weight of the cis-9, trans-11 CLA or salt or ester thereof and the VA or salt or ester thereof.

10. A pharmaceutical composition of claim 7, wherein the composition comprises about 5% to about 30% by weight cis-9, trans-11 CLA or a salt or ester thereof and about 95% to about 70% by weight VA or a salt or ester thereof based on the combined weight of the cis-9, trans-11 CLA or salt or ester thereof and the VA or salt or ester thereof.

11. A pharmaceutical composition of claim 10, further comprising one or more agents selected from bronchodilators, anticholinergic agents and anti-inflammatory agents.

12.-23. (canceled)

24. A method of treating or preventing a condition associated with one or more of leukocyte infiltration, eosinophilia, airway remodelling, bronchoconstriction, mucus hypersecretion, lung inflammation and skin inflammation, the method comprising administering cis-9, trans-11 conjugated linoleic acid (CLA) or a salt or ester thereof and vaccenic acid (VA) or a salt or ester thereof separately, simultaneously or sequentially to a subject in need thereof.

25. A method of claim 24, comprising administration of a composition comprising about 5% to about 30% by weight cis-9, trans-11 conjugated linoleic acid (CLA) or a salt or ester thereof and about 95% to about 70% by weight vaccenic acid (VA) or a salt or ester thereof.

26. A method of claim 25, wherein the condition is selected from atopic conditions, eosinophilias and Th2-mediated conditions.

27. A method of claim 25, wherein the condition is asthma.

28. A method of claim 25, wherein the condition is dermatitis.

29. A method of claim 25 for treating or preventing with steroid sparing effect a condition associated with one or more of leukocyte infiltration, eosinophilia, airway remodelling, bronchoconstriction, mucus hypersecretion, and lung and skin inflammation.

30. (canceled)

31. A method of claim 29, wherein the condition is selected from asthma, eczema, eosinophilic gastroenteritis, eosinophilic pneumonia and hyper-eosinophilic syndrome.

32. A method of claim 24, further comprising separate, simultaneous or sequential administration of bronchodilator, an anticholinergic agent, an anti-inflammatory agent, or a combination thereof.

33-37. (canceled)
